(19) United States
(12) Patent Application Publication
(10) Pub. No.: US 2014/0259629 A1
(43) Pub. Date: Sep. 18, 2014

(54) DEVICES, METHODS AND SYSTEMS FOR FORMING IMPLANT COMPONENTS

(71) Applicant: CONFORMIS, INC., Bedford, MA (US)

(72) Inventors: Ernest Dion, Danvers, MA (US); Bob Miller, Secaucus, NJ (US); Phil Pereira, Wilmington, MA (US); David Hesketh, Methuen, MA (US); Manuel J Salinas, North Andover, MA (US); Philipp Lang, Lexington, MA (US)

(73) Assignee: CONFORMIS, INC., Bedford, MA (US)

(21) Appl. No.: 14/216,473

(22) Filed: Mar. 17, 2014

Related U.S. Application Data
(60) Provisional application No. 61/799,298, filed on Mar. 15, 2013.

(51) Int. Cl. A61F 2/30; A61F 2/38

(52) U.S. Cl. CPC A61F 2/30942; A61F 2/3859
USPC 29/558

(57) ABSTRACT

Patient-specific implants and implant components, as well as methods of making patient-specific implants and implant components are disclosed herein. In particular, various embodiments include making a patient-specific implant component utilizing an electrical discharge machining technique.

### RELATED APPLICATIONS

This application claims the benefit of U.S. Provisional Application No. 61/799,298, entitled “Devices, Methods and Systems for Forming Implant Components” and filed Mar. 15, 2013, the disclosure of which is incorporated herein by reference in its entirety.

### TECHNICAL FIELD

The embodiments described herein relate to devices, methods and systems for manufacturing implants, implant components and/or related tools using electrical discharge machining (EDM) or similar manufacturing techniques to manufacture implant components for artificial joints. More specifically, various embodiments described herein include methods for improving the manufacture and/or modification of joint replacement and/or resurfacing components that utilize a partially-manufactured blank component to create patient-specific femoral implant components via a variety of manufacturing methods, including the use of wire EDM and/or related machining techniques.

### BACKGROUND

Historically, diseased, injured or defective joints, such as, for example, joints exhibiting osteoarthritis, were repaired using standard off-the-shelf implants and other surgical devices. Surgical implant systems that employed a one-size-fits-all approach to implant design (and even those that utilized a “few-sizes-fit-all” approach, including modularly assembled systems) did not typically require highly accurate information about the patient's anatomy. Instead, such systems utilized gross anatomical measurements such as the maximum bone dimensions at the implant site, as well as the patient weight and age, to determine a “suitable” implant. The surgical procedure then concentrated on altering the underlying bony anatomical support structures (i.e., by cutting, drilling and/or otherwise modifying the bone structures) to accommodate the existing contact surfaces of the pre-manufactured implant. With these systems, varying quantities of implants and/or implant components could be manufactured in large quantities and stockpiled. Once a potential patient was identified, an appropriate implant and/or component would be selected, transported to the surgical location and utilized in the patient's surgical procedure.

More recently, “patient-specific” and “patient-engineered” implant systems have been developed that benefit from new manufacturing methods, for example to improve the quality of individual devices and components as well as to improve the efficiency in the manufacturing process and reduce cost. With such systems, the surgical implants, associated surgical tools and procedures are designed or otherwise modified to account for and accommodate the individual anatomy of the patient undergoing the surgical procedure. Such systems typically utilize non-invasive imaging data, taken of the individual pre-operatively, to guide the design and/or selection of the implant, surgical tools, and the planning of the surgical procedure itself.

A number of challenges exist in the development, design and manufacture of patient-specific implants and associated surgical procedures, many of which relate to the time and expense required to manufacture a unique implant for each individual surgical patient. Unlike standard and/or modular implants, which can be cast in bulk quantities and stored/stockpiled for use as needed, patient-specific implants are generally created after a patient has been identified as a surgical candidate, and the implant components are designed and/or selected using imaging data taken of the intended patient's anatomy. In some cases, traditional methods of creating of a patient-specific implant from patient imaging data can require several weeks and cost a significant amount per implant.

Another factor affecting the design and manufacture of patient-specific implants relates to the potential for processing-related failures that may occur during the manufacture of the patient-specific implant components. Moreover, traditional implant manufacturing typically involves “heavy” and large scale manufacturing equipment and processes that are not efficient or appropriate for the creation of single implants. Because “patient-specific” and “patient-engineered” implant systems are not pre-manufactured and stockpiled in multiple sizes (as are traditional systems), there can be additional manufacturing time associated with such devices and systems. Typically, such implant components are manufactured using various combinations of traditional casting techniques (i.e., designing and creating a mold, and then filling the mold with molten material that cools and hardens into a desired shape) and machining techniques (i.e., machining a casting or bulk material stock to a desired shape using subtracting machining processes such as drilling, cutting, milling, lathing, abrading, etc.). Such traditional manufacturing techniques, when undertaken for the manufacture of small batches or individual implants, can increase the cost and time of creating such patient-specific implant components as compared to the large batch manufacturing used with traditional non-custom implants. In addition, because “patient-specific” and/or “patient-engineered” implant systems are manufactured in limited quantities, a fracture, failure or sufficient discrepancy identified at any point in the manufacturing process can have significant consequences, including the non-availability of implant components when needed, a requirement to remanufacture implant components, and/or the need to order implant components on an expedited basis to meet deadlines or the rescheduling of the surgery, which can add cost and be more expensive than manufacturing implants on a regular basis.

Implant manufacturers also desire to establish “backup options” to guarantee an implant component is properly processed and available for a given surgical procedure. Since each patient-specific implant is unique, and a significant amount of time and effort is typically required to create each implant. One method of avoiding the adverse impact of an implant or instrument component that does not pass inspection or “falls out” of the manufacturing process for other reasons is to create a second “backup” component, to ensure implant availability by the promised date for a given surgical procedure. This back-up option process can ensure that at least one patient-specific implant survives the manufacturing, finishing and testing processes prior to surgical use. However, this adds additional cost.

Accordingly, there is a need for improved methods, techniques, devices and systems for the design and manufacture of “patient-specific” and/or “patient-engineered” implant components, as well as to improve and support other operational aspects in the field.

### SUMMARY

The embodiments described herein include advancements and improvements in or related to the use of electrical discharge machining or “EDM” manufacturing or similar manufacturing techniques in the design, selection, development, manufacturing and/or finishing of patient-specific and/or patient-engineered implant components. Various embodiments described herein facilitate the production of “patient-specific” or “patient-engineered” implants in a more cost effective and/or efficient manner than traditional casting and/or machining techniques.

Various embodiments described herein include methods for improving the strength, quality, performance and/or durability of implant components manufactured using EDM or similar manufacturing techniques.

Various embodiments described herein include methods of improving and/or simplifying the post-manufacture processing and/or “finishing” of an implant component manufactured using EDM or similar manufacturing techniques.

Various embodiments described herein include methods of assessing and/or optimizing EDM manufacturing methods and/or modifying implant design features to accommodate different limitations associated with EDM manufacturing techniques and processes.

It is to be understood that the features of the various embodiments described herein are not mutually exclusive and may exist in various combinations and permutations.

### BRIEF DESCRIPTION OF THE SEVERAL VIEWS OF THE DRAWINGS

FIG. 1 depicts an isometric view of one preferred embodiment of a femoral blank that can be used in an EDM and/or machining process to manufacture a patient-adapted implant component;

FIG. 2 depicts various plan views of a right femur, illustrating various approximated dimensions that could be used in selecting and/to designing an appropriately-sized femoral blank;

FIG. 3A depicts a front view of the femoral blank of FIG. 1;

FIG. 3B depicts the femoral blank of FIG. 3A, overlain with an implant component design profile;

FIG. 4 depicts a side view of the embodiment of FIG. 3A;

FIG. 5 depicts an isometric view of an EDM fixture that can be used to skin or “true up” the supports posts of the femoral blank of FIG. 1;

FIG. 6A depicts an isometric view of a material layer removed during a “skin cut” step;

FIG. 6B depicts a partial perspective view of an implant blank after removal of the material layer skin of FIG. 6A;

FIG. 7 depicts an isometric view of a second EDM fixture that integrates with the skinned support posts of the femoral blank;

FIG. 8A depicts an isometric view of a material layer removed from the femoral blank of FIG. 5 during a subsequent EDM cutting operation;

FIG. 8B depicts an isometric view of the femoral implant blank remaining after removal of the material layer of FIG. 8A;

FIG. 9 depicts a side view of another EDM fixture that integrates with the skinned support posts of the femoral blank;

FIG. 10 depicts a side view of a posterior profile cut plane for a femoral implant using the EDM fixture of FIG. 9;

FIG. 11 depicts a side view of an anterior profile cut plane for a femoral implant using the EDM fixture of FIG. 9;

FIG. 12 depicts an isometric view of one preferred embodiment of a femoral implant blank after initial shaping using a wire EDM process;

FIG. 13 depicts a side view of the femoral implant blank of FIG. 12;

FIG. 14 depicts a bottom view of the femoral implant blank of FIG. 12;

FIG. 15 depicts a posterior/anterior view of the femoral implant blank of FIG. 12;

FIG. 16 depicts a top plan view of the femoral implant blank of FIG. 12;

FIG. 17 depicts an isometric view of a trunnion fixture that can be used for CNC machining of an implant after initial EDM processing;

FIG. 18 depicts an side plan view of the trunnion fixture of FIG. 17;

FIG. 19 depicts an isometric view of a securing fixture that can be used to support a femoral implant component for machining after initial EDM processing;

FIGS. 20 through 23 depict various views of the securing fixture of FIG. 19 holding a femoral implant blank after initial EDM processing;

FIG. 24 depicts an isometric view of an embodiment of a securing fixture for holding a femoral implant component after machining of the inner surface;

FIG. 25 depicts a top plan view of the securing fixture of FIG. 24;

FIG. 26 depicts a side view of the securing fixture of FIG. 24;

FIGS. 27 through 29 depict various views of the securing fixture of FIG. 24 with an attached femoral implant blank;

FIGS. 30 through 32 depict various views of a fixture block and hydraulic vise arrangement for securing multiple femoral implants within a single piece processing machine;

FIG. 33 depicts a top view of a bone model incorporating patient-specific anatomical and surgical procedural data that can be manufactured and used to inspect a finished femoral implant;

FIG. 34 depicts a side view of the bone model and finished femoral implant of FIG. 33; and

FIGS. 35A through 35D depict views of a final finished patient-specific implant component manufactured using various of the EDM and machining techniques described herein.

### DETAILED DESCRIPTION

In this application, the use of the singular includes the plural unless specifically stated otherwise. Furthermore, the use of the term “including,” as well as other forms, such as “includes” and “included,” is not limiting. Also, terms such as “element” or “component” encompass both elements and components comprising one unit and elements and components that comprise more than one subunit, unless specifically stated otherwise. Also, the use of the term “portion” may include part of a moiety or the entire moiety.

The section headings used herein are for organizational purposes only and are not to be construed as limiting the subject matter described or the combination of features and/or embodiments described under one heading with features and/or embodiments described under another heading.

Various exemplary embodiments include devices, systems and methods for manufacturing patient-specific and/or patient-adapted implant components. Various of the exemplary methods disclosed herein include the use of a limited quantity of pre-manufactured and/or partially-manufactured “blanks” that can be manufactured using traditional bulk manufacturing techniques and stockpiled for use, and which are then quickly selected and modified into patient-specific implant components appropriate for implantation into a given patient and/or patient group.

### Implant Blanks

In at least one exemplary embodiment, a series of implant blanks can be designed, manufactured and stockpiled, such as an inventory of blanks in small, medium and large sizes. These standard sizes may be derived using a set standard size and/or shape of implant component (or size/shape ranges thereof), using patient-specific images or from a database library. The various sized blanks can accommodate a wide variety of potential implant component shapes and sizes, such that a significant portion of the anticipated patient-specific implant component designs suitable for a given patient population can be created out of the various blanks.

For example, pre-manufactured blank sizes and/or dimensions thereof can be derived using one or more patient-specific images and/or image data for one or more patients or patient populations, which can provide highly accurate dimensions and surface/subsurface feature measurements of appropriate surgical implant components that define a desired range of implant blank dimensions. The images or image data sources can be based on three-dimensional (3D) images or two dimensional (2D) images, or sets of two-dimensional images ultimately yielding 3D information on a patient or patient population. Two-dimensional and three-dimensional images, or maps, of the particular joints, and/or any such data in combination with movement patterns of the joint, e.g. flexion-extension, translation and/or rotation, can be obtained as source data. 2D images can include information on movement patterns, contact points, contact zones of two or more opposing articular surfaces, and movement of the contact point or zone(s) during joint motion. In addition, imaging techniques can be compared over time, for example to provide up-to-date information on the shape and types of material needed.

In one exemplary embodiment, a desired range of femoral implant component features may be derived and/or selected using measurements of thickness, size, area, volume, width, perimeter and/or surface contour of the diseased femur or other joints obtained from a reference population or from a database library, where the data collected from the reference population may be stored in a database which can be periodically or continuously updated. The dimensional ranges and features of appropriate or exemplary femoral implant component can be derived and/or selected using the captured measurements from the referenced population or various patient-specific or patient-engineered measurements can be correlated to the reference population database to predict measurements, shapes or contours that may be necessary for optimal sizing of the implant component. In various other embodiments, a series of pre-existing implant designs from a database (i.e., from a series of ConforMIS implants previously used to treat various patients or patient populations) can be queried to identify desired sizes, shapes and/or configurations of implant blanks and blank sets.

Once a given range or ranges of anticipated implant sizes, shapes and/or other features has been determined, a series of implant blank shapes and sizes can be derived to accommodate the range(s), each blank in the series sized to accommodate a portion of the desired range of sizes. In various cases, implant components for unusual or highly deformed patient anatomy may not be accommodated by a given range of blank sizes (i.e., “outliers”), but the chosen dimensions of the various blank sizes can permit a significant portion of the anticipated implant component to be manufactured from the one or more blanks in the series. Once the desired blank dimensions have been determined, a quantity of the different sized blanks may be manufactured using standard manufacturing techniques (i.e., casting or forging of blanks in large quantities a volume pricing) and stockpiled for use as needed.

In various cases, the measurements of anticipated implant components can be pre-selected or otherwise “driven” such that they reflect measurement or features from a reference population or database library that was used to design an implant blank assembly closely matching at least one or more of these measurements. In such cases, the design of a given implant component feature may be selected from a variety of acceptable alternatives (i.e., feature sizes and/or shapes) to approximate features that can be accommodated by the readily-available blanks (i.e., blanks already designed, manufactured and warehoused), with the blanks subsequently processed to the desired more exacting size and/or shape for use in the targeted patient.

FIG. 1 depicts an isometric view of one exemplary embodiment of a femoral implant blank 10. Although a femoral implant blank is depicted, the various teachings described herein can be applied with equal utility to implant components and joint implant blanks designed and manufactured for other damaged or diseased articulating joints, such as the ankle, wrist, shoulder, hip, finger, toe and/or vertebrae (i.e., including the intervertebral discs, costovertebral joints, contravertebral joint and/or facet joints).

Implant blanks may be manufactured using a variety of materials, including those that may facilitate or reduce the manufacturing time and/or commercialization for various joint-specific and/or loading requirements. Various materials contemplated can include materials that are known and used in the medical device industry; for example, the implant blank may be formed from a wide variety of biomedical and/or biocompatible materials, including materials that exhibit superior properties for their intended use, such as high performance polyethylenes, low friction polymers, titanium, stainless steel, flexible materials or hybrid of biomaterial combinations. The strength, weight, and/or sterilization requirements can be considered in designing and selecting the various features of the implant blank.

FIG. 2 illustrates various plan views of a right femur 20, which highlight various anatomical dimensions that a manufacturer may consider when sizing and designing a set of joint implant blanks. For example, a given size and shape of a femoral implant blank will include sufficient material to accommodate a variety of femoral implant component sizes and shapes, which in turn can typically be derived using various dimensions and other anatomical features from a patient's image file, which could include such measurements as their condyle width 40, the condyle height 50, condyle depth 30, condylar curvatures and surface features, opposing joint surface features and/or other measurements.

In one exemplary embodiment, the various dimensions for one or more blanks can be derived directly from anatomical data containing a large number of anatomical feature measurements and/or image data from a variety of patients from a given patient population which has been entered into a database library. From this database, various derived ranges of anatomical measurements can be determined, which a designer or manufacturer can use to design and manufacture one or more implant blanks appropriate to the manufacture of implant components that accommodate such measurement ranges. Once a prospective patient has been identified, and relevant patient measurements and/or image data has been acquired, the patient measurements and/or image data can be compared to the available blank dimensions and/or to the measurement range data and used to select an appropriately sized implant blank for further processing.

In one alternative embodiment, the various dimensions for one or more blanks can be derived from dimensional data of a large number of implant components (preferably from a similar type of implant component) that were previously designed for a variety of patients from a given patient population, with such component data having been entered into a database library. From this database library, various desired ranges of component measurements can be determined, which a designer or manufacturer can use to design and manufacture one or more implant blanks appropriate to the manufacture of implant components that accommodate such ranges.

FIG. 3A illustrates a front view of the exemplary femoral blank 10 of FIG. 1. In this embodiment, the femoral blank includes a central body 85, a first raised section 62, a second raised section 82 and one or more extrusions or support stems 140. The raised sections 62 and 82 can be of similar heights and/or thicknesses, or such dimensions may be different, depending upon the chosen blank design. In the disclosed embodiment, the raised sections are of differing thicknesses, with the first raised section 62 having a first width 60 that is less than a second width 80 of the second section 82. The raised sections 62 and 82 also incorporate differing heights, with the first raised section 62 having a first height 64 and the second raised section 82 having a second height 84.

The differing dimensions of the blank in general, and the raised sections in particular, can be derived and/or selected using various anatomical and/or implant dimensional datasets and/or various manufacturing parameters to design one or more blanks suited for use in creating the desired implant components. The blank can accommodate the creation of implant component of differing shapes, sizes and/or configurations, and the finished implant could incorporate a significant variety of component feature combinations.

For example, FIG. 3B depicts an implant blank 10 overlain with a profile of an implant component design 700. The component design 700 includes an anterior section 705 and a posterior section 710, with a height 715 of the anterior section 705 being significantly less prominent than a height 720 of the posterior section 710. The differential heights of the first and second raised sections 62 and 82 of the blank 10 accommodate the differing heights of the component design 700, yet require a limited amount of material to be removed from the blank to replicate the profile design.

The thicknesses of the raised sections 62 and 82 can be selected and/or designed to accommodate a variety of features and/or dimensional variation in the implant components to be manufactured therefrom. For example, the raised section 82 can accommodate the various locations that the distal ends of the medial and lateral posterior condylar surfaces can occupy, as well as provide sufficient material thicknesses to manufacture such implant sections. In addition, where such surfaces are asymmetric and/or offset (i.e., the various surfaces of the two structures do not occupy the same medial/lateral planes), the blank design can accommodate the manufacture of such surfaces. In many cases, the features of the implant may closely match the native condyle measurements to reflect natural or native alignment, rotation, and movement.

Another significant feature of the implant blank 10 of FIG. 3A is the width 70 of the blank between raised sections 62 and 82 as well as the depth 100 of the central body 85. The blank width 70 accommodates a variety of implant shapes and sizes, such that the amount of material removal that is required from the open region 88 between the raised sections 62 and 82 is significantly reduced as compared to a block of raw material stock (i.e., the material in open region 88 has already been removed in the blank manufacturing process).

Because the design of a femoral implant component often requires the use of one or more bone-anchoring pegs (not shown), the depth 100 of the central body is sufficiently thick to allow creation of one or more pegs integrally with the inner, bone-facing surface of the blank (i.e., a portion of the central body 85 facing towards the open region 88). These pegs are integrally formed with the central portion of the implant component, although attachable pegs or other features could be used in alternative embodiments. Because the placement of pegs can vary widely on the surfaces of the implant, and the use of two or more pegs is typically desired, the blank design allows placement of such pegs in almost any position relative to the condyles of the implant. A lateral profile of the pegs can be cut, and then the individual pegs later formed in a subsequent machining operation.

The exemplary blank design can be used to manufacture a variety of implant component designs and features. An electronic representation of the various dimensions and features of the blank 700 can be contained in a database or other computing equipment, and in a preferred embodiment a plurality of such blanks, and the associated electronic representations thereof, will be stored in a similar manner. Once an intended implant component design for a specific patient and/or patient population has been determined and/or selected using patient-specific anatomical information and/or other data sources, a computing device can compare the intended implant component design to one or more of the electronic representations of the various available blanks to identify one or more blanks that can be utilized to manufacture the final implant component.

In various embodiments, the computing device can include programming features that facilitate 3-dimensional manipulation of the intended implant component design and/or the electronic blank representation(s) to merge, match and/or otherwise determine whether a given blank could be utilized to manufacture the implant component. For example, the electronic representation of the exemplary blank could be digitally manipulated and rotated in three dimensions to identify whether the intended implant component design could fit completely within the boundaries of the electronic blank representation. Various algorithms, such as packing algorithms, could be employed to determine the suitability of a given blank relative to a given implant component design. If a first electronic blank representation is identified as unsuitable and/or not available in inventory, then the computing device could move on to comparing other electronic blank representations for blanks of other sizes and/or shapes to the intended implant component design for potential matches.

FIG. 4 depicts a side view of the femoral blank 10 of FIG. 1, highlighting individual support posts 140 and 142 that are initially formed as part of an implant blank 10. The support posts 140 can be used as securement and/or alignment guides that assist with several of the manufacturing processes, such as EDM cutting or machining. In manufacturing the implant from a blank, the various manufacturing steps and processes typically require that one or more reference points on the implant can be accurately determined to allow accurate machining and/or forming of the implant, thereby facilitating the creation of the various surfaces of the implant free from deformity, defects and/or abnormalities. The support posts 140 can be used to hold the implant blank during the various processing steps described herein, such as EDM or machining, and further act as datum so as to ensure accurate processing and creation of the various surface features.

Once a manufacturer has determined the specific sizes and/or shapes of implant blanks it wishes to produce and store in inventory, the manufacturer can select an appropriate blank based on the implant component designs intended for a desired surgical procedure. Alternatively, an appropriate implant blank may merely be designated as a “back-up” patient-specific implant, where the primary implant is manufactured via other techniques, including standard manufacturing techniques. Where manufacture of an implant from the blank is desired, the selected implant blank will then undergo further combinations of manufacturing techniques, including wire EDM and machining, and then the final component can be finished, polished, packaged and shipped for use in a surgical procedure. In various embodiments, the required manufacturing time from implant design to finished implant component can be reduced from 4 to 6 weeks to a matter of a few hours and/or days.

### Manufacturing Techniques

Various technologies appropriate for manufacturing implants and tools are known in the art, for example, as described in Wohlers Report 2009, State of the Industry Annual Worldwide Progress Report on Additive Manufacturing, Wohlers Associates, 2009 (ISBN 0-9754429-5-3), available from the web www.wohlersassociates.com; Pham and Dimov, Rapid manufacturing, Springer-Verlag, 2001 (ISBN 1-85233-360-X); Grenda, Printing the Future, The 3D Printing and Rapid Prototyping Source Book, Castle Island Co., 2009; Virtual Prototyping & Bio Manufacturing in Medical Applications, Bidanda and Bartolo (Eds.), Springer, Dec. 17, 2007 (ISBN: 10: 0387334297; 13: 978-0387334295); Bio-Materials and Prototyping Applications in Medicine, Bártolo and Bidanda (Eds.), Springer, Dec. 10, 2007 (ISBN: 10: 0387476822; 13: 978-0387476827); Liou, Rapid Prototyping and Engineering Applications: A Toolbox for Prototype Development, CRC, Sep. 26, 2007 (ISBN: 10: 0849334098; 13: 978-0849334092); Advanced Manufacturing Technology for Medical Applications: Reverse Engineering, Software Conversion and Rapid Prototyping, Gibson (Ed.), Wiley, January 2006 (ISBN: 10: 0470016884; 13: 978-0470016886); and Branner et al., “Coupled Field Simulation in Additive Layer Manufacturing,” 3rd International Conference PMI, 2008. While many of these described technologies have the potential to assist the implant manufacturer in reducing the time to build a patient-specific implant by maximizing productivity, accelerate product development and design, the selection of an appropriate manufacturing technology and/or combinations thereof can be a difficult task. Use of only a single technology may not enable creation of an implant in a timely an accurate manner, and the various limitations inherent in each manufacturing technique may result in design and/or manufacturing errors and issues that are only discovered later during an implant inspection. In many cases, the appropriate use of a combination of manufacturing techniques, such as described herein, can facilitate the rapid manufacturing of a custom implant from an implant blank to save time, money, and potentially produce a higher quality custom implant.

In many cases, the most appropriate combination of manufacturing technologies to produce a patient-specific implant can depend on a variety of factors, including the implant's function, the material used, time, cost and available equipment and trained manufacturing personnel. The table below describes many manufacturing technologies that may be used and combined for rapid manufacturing in the various methods described herein.

Exemplary techniques for forming or altering a patient-specific and/or patient-engineered implant component for a patient's anatomy

| Technique | Brief description of technique and related notes |
|---|---|
| CNC | CNC refers to computer numerically controlled (CNC) machine |
| | tools, a computer-driven technique, e.g., computer-code |
| | instructions, in which machine tools are driven by one or more |
| | computers, Embodiments of this method can interface with |
| | CAD software to streamline the automated design and |
| | manufacturing process. |
| CAM | CAM refers to computer-aided manufacturing (CAM) and can |
| | be used to describe the use of software programming tools to |
| | efficiently manage manufacturing and production of products |
| | and prototypes. CAM can be used with CAD to generate CNC |
| | code for manufacturing three-dimensional objects. |
| Casting, including | Casting is a manufacturing technique that employs a mold. |
| casting using rapid | Typically, a mold includes the negative of the desired shape of |
| prototyped casting | a product. A liquid material is poured into the mold and |
| patterns | allowed to cure, for example, with time, cooling, and/or with |
| | the addition of a solidifying agent. The resulting solid material |
| | or casting can be worked subsequently, for example, by |
| | sanding or bonding to another casting to generate a final |
| | product. |
| Welding | Welding is a manufacturing technique in which two |
| | components are fused together at one or more locations. In |
| | certain embodiments, the component joining surfaces include |
| | metal or thermoplastic and heat is administered as part of the |
| | fusion technique. |
| Forging | Forging is a manufacturing technique in which a product or |
| | component, typically a metal, is shaped, typically by heating |
| | and applying force. |
| Rapid prototyping | Rapid prototyping refers generally to automated construction |
| | of a prototype or product, typically using an additive |
| | manufacturing technology, such as EBM, SLS, SLM, SLA, DMLS, |
| | 3DP, FDM and other technologies |
| EBM ® | EBM ® refers to electron beam melting (EBM ®), which is a |
| | powder-based additive manufacturing technology. Typically, |
| | successive layers of metal powder are deposited and melted |
| | with an electron beam in a vacuum. |
| SLS | SLS refers to selective laser sintering (SLS), which is a powder- |
| | based additive manufacturing technology. Typically, |
| | successive layers of a powder (e.g., polymer, metal, sand, or |
| | other material) are deposited and melted with a scanning |
| | laser, for example, a carbon dioxide laser. |
| SLM | SLM refers to selective laser melting ™ (SLM), which is a |
| | technology similar to SLS; however, with SLM the powder |
| | material is fully melted to form a fully-dense product. |
| SLA or SL | SLA or SL refers to stereolithography (SLA or SL), which is a |
| | liquid-based additive manufacturing technology. Typically, |
| | successive layers of a liquid resin are exposed to a curing, for |
| | example, with UV laser light, to solidify each layer and bond it |
| | to the layer below. This technology typically requires the |
| | additional and removal of support structures when creating |
| | particular geometries. |
| DMLS | DMLS refers to direct metal laser sintering (DMLS), which is a |
| | powder-based additive manufacturing technology, Typically, |
| | metal powder is deposited and melted locally using a fiber |
| | optic laser. Complex and highly accurate geometries can be |
| | produced with this technology. This technology supports net- |
| | shaping, which means that the product generated from the |
| | technology requires little or no subsequent surface finishing. |
| LC | LC refers to LaserCusing ® (LC), which is a powder-based |
| | additive manufacturing technology. LC is similar to DMLS; |
| | however, with LC a high-energy laser is used to completely |
| | melt the powder, thereby creating a fully-dense product. |
| 3DP | 3DP refers to three-dimensional printing (3DP), which is a high- |
| | speed additive manufacturing technology that can deposit |
| | various types of materials in powder, liquid, or granular form in |
| | a printer-like fashion. Deposited layers can be cured layer by |
| | layer or, alternatively, for granular deposition, an intervening |
| | adhesive step can be used to secure layered granules together |
| | in bed of granules and the multiple layers subsequently can be |
| | cured together, for example, with laser or light curing, |
| LENS | LENS ® refers to Laser Engineered Net Shaping ™ (LENS ®), which |
| | is a powder-based additive manufacturing technology. |
| | Typically, a metal powder is supplied to the focus of the laser |
| | beam at a deposition head. The laser beam melts the powder |
| | as it is applied, in raster fashion. The process continues layer |
| | by and layer and requires no subsequent curing. This |
| | technology supports net-shaping, which means that the |
| | product generated from the technology requires little or no |
| | subsequent surface finishing. |
| FDM | FDM refers to fused deposition modeling ™ (FDM) is an |
| | extrusion-based additive manufacturing technology. Typically, |
| | beads of heated extruded polymers are deposited row by row |
| | and layer by layer. The beads harden as the extruded polymer |
| | cools. |
| EDM | EDM refers to electric discharge machining (EDM) where a |
| | desired shape is obtained using a series of rapidly recurring |
| | electrical discharges between two electrodes. Various EDM |
| | processing techniques are available to accommodate the type |
| | of material. |

### Accommodating Different Manufacturing Methods

Implant components generated by different manufacturing techniques can be assessed and compared for their accuracy of shape relative to the intended shape design, for their mechanical strength, the type of material, cost and for other factors. In this way, different manufacturing techniques can supply another consideration for achieving an implant component design with one or more target features. For example, if accuracy of shape relative to the intended shape design is important to a particular patient's implant component design, then the manufacturing technique supplying the most accurate shape may be selected. If a minimum implant thickness is important to a particular patient's implant component design, then the manufacturing technique supplying the highest mechanical strength and therefore potentially allowing the most minimal implant component thickness, can be selected. Branner et al. describe a method for the design and optimization of additive layer manufacturing through a numerical coupled-field simulation, based on the finite element analysis (FEA). Branner's method can be used for assessing and comparing product mechanical strength generated by different additive layer manufacturing techniques, for example, SLS, SLM, DMLS, and LC.

In certain embodiments, an implant can include components and/or implant component parts produced via various methods. For example, in certain embodiments for a knee implant, the knee implant can include a metal femoral implant component produced by casting or by an additive manufacturing technique and having a patient-specific femoral intercondylar distance; a tibial component cut from a blank and machined to be patient-specific for the perimeter of the patient's cut tibia; and a tibial insert having a standard lock and a top surface that is patient-specific for at least the patient's intercondylar distance between the tibial insert dishes to accommodate the patient-specific femoral intercondylar distance of the femoral implant.

As another example, in certain embodiments a knee implant can include a metal femoral implant component produced by casting or by an additive manufacturing technique that is patient-specific with respect to a particular patient's M-L dimension and standard with respect to the patient's femoral intercondylar distance; a tibial component cut from a blank and machined to be patient-specific for the perimeter of the patient's cut tibia; and a tibial insert having a standard lock and a top surface that includes a standard intercondylar distance between the tibial insert dishes to accommodate the standard femoral intercondylar distance of the femoral implant.

In a further example, a patient-specific knee implant or any other joint implant can manufactured by using a blank implant template from inventory that is patient-specific with respect to a particular patient's M-L dimension and standard with respect to the patient's femoral intercondylar distance; the implant may undergo EDM to cut approximately specifically shaped contours and cavities on both the proximal and articulating side of the implant; and the implant can subsequently undergo further machining on a CNC to substantially match or match the desired dimensions of the implant components and/or desired patient's dimensions of the joint.

### Various Electric Discharge Machining Techniques (EDM)

Although there are a variety of combinations of manufacturing methods that can potentially rapidly produce implants, the EDM process is a method of making prototype and production implants in which production quantities are relatively low and accuracy of cut (i.e. patient specific implants) is desired. There are many types of EDM techniques which can be selected, and such selection is typically based primarily on a variety of manufacturing parameters that the manufacturer may be interested in. EDM may be used to machine materials that are electrically conductive. In EDM, a potential difference is generated between an electrode of the EDM machine and the work piece. The potential difference between the electrode and the work piece causes a spark to be generated. The spark erodes a portion of the work piece, and consecutive sparks between the electrode and the work piece are used to remove material from the work piece. Because the electrode may also be damaged by the spark, the electrode is typically continuously replaced. For example, in EDM using wire electrodes, the electrode wire is continuously advanced while the work piece is being fabricated. The work piece may be shapes by moving the work piece relative to the electrode, moving the electrode relative to the work piece, or various combinations thereof. For example, spherical and curved shapes may be formed using EDM machinery by rotating the work piece while the electrode is moved along an arc.

For example, one EDM technique is known as the basic or conventional EDM process (or ram or die-sinking EDM), in which a graphite electrode is machined into a desired shape and mounted onto the end of a vertical ram. Power is applied to the electrode, and an electrical spark is generated between the electrode and a surface of the implant in close proximity to the electrode. The electrical spark created is quite visible and usually produces intense heat reaching 8,000 to 12,000 degrees Celsius, which can melt or erode any material that may be placed in front of it. To assist with conductivity of the spark, a dielectric deionized water can be provided between the electrode and implant, with the liquid providing an excellent environment for conductivity, functionality as a coolant and an ability to flush away the eroded metal particles. In this process, the inverted image of the graphite tool electrode can be gradually impressed in the implant.

Another exemplary EDM technique is EDM wire cutting, which involves the use of a thin, single strand of metal wire that has an electrical discharge current running through it. The wire is constantly fed from a spool during cutting, and the cutting also occurs in a dielectric fluid (i.e., a water bath that can control resistivity and/or conductivity, and also act as a coolant and flushing medium). The cutting path for a typical wire set-up is along a straight path, and the path diameter can be as small as 0.021 mm (which can be accomplished by a 0.02 mm diameter wire). The cutting width of the path is typically slightly larger (i.e., the erosion creates a “kerf” path slightly larger than the wire) because the electrical sparking emitted from the wire to the implant causes erosion between the implant and wire, and the wire does not physically contact the implant. The cut path dimension of wire EDM is quite predictable and can be compensated by using smaller wire diameters to achieve the desired dimension. Micro wires may also be used, and may be as small at 20 micrometers, and the precision does not deviate far from +/−1 micrometer.

In addition to cutting parts along a fixed axis, wire EDM techniques may also integrate features such as multi-axis EDM wire cutting for cutting multiple parts at the same time, to cut curved surfaces (i.e., by moving the work piece along a desired rotational and/or curved path relative to the wire), and/or to cut very intricate and delicate shapes. In various embodiments, a wire can be inclined to make it possible to make parts with a taper or different profiles for the superior or posterior surface of implants.

With such desired tighter tolerances on the cuts, the precision of the cuts, and the quality of the surface finish using wire EDM, this technique allows an implant component to be initially “roughed”—producing relatively large scrap pieces from the initial implant component after this initial cutting step has taken place. A subsequent skim cut by wire EDM may then be performed at a lower power setting and/or with a lower pressure flush, which can give a high quality surface and/or more accurate desired shape. The manufacturer may choose the accuracy and the surface finish by performing one or multiple skim passes.

Another EDM related process is electrical discharge milling (EDMG), which uses standard cylindrical rotating graphite electrodes to produce electrical sparks that can affect material of a work piece in a manner similar to physical milling. A desired shape may occur after successive passes of the electrode over the implant until the cut achieves the desired depth. The use of standard graphite electrodes using this technique can significantly reduce the cost of making expensive, complex electrode shapes.

A fourth type of EDM process is known as Rotary EDM or EDM Grinding, which uses a rotating electrically conductive wheel (similar in size to a standard abrasive grinding wheel) as the tool electrode to perform electrical discharge erosion similar to creep-feed grinding.

Another type of EDM process is known as electrical discharge dressing (EDD), which uses the electrical discharge erosion effect to modify devices during use, such as dressing grinding wheels in real-time when mechanically grinding tough materials. One limitation of this technique is that the grinding wheel is electrically conductive (for example, a metal bonded diamond grinding wheel can be dressed by this method). In the technique, a pulsed electrical voltage is applied between the electrode and the grinding wheel or other work piece in which the generated electrical discharge removes the built-up edges on the grinding wheel.

Another type of EDM process is ultrasonic aided EDM (UEDM), which includes a thermal material removal process in which material is removed by electrical discharge erosion with a tool electrode that is vibrating at ultrasonic frequency. The ultrasonic vibration can significantly improve the machining stability and substantially increase machining rates when drilling small or micro holes.

Another type of EDM process is Abrasive Electrical Discharge Grinding (AEDG), which is a hybrid process in which material is removed by a combination action of the electrical discharge erosion and mechanical grinding for machining advanced ultra-hard materials. This process is particularly useful for machining polycrystalline diamond (PCD) materials, but can also be useful in processing other relatively hard materials. Electrical discharges help to increase the material removal rate and the mechanical grinding can generate a fine surface finish.

Another type of EDM process is Micro Electrical Discharge Machining (MEDM), which can include miniature sinker type machines or wire electrodes utilizing a diamond V-groove to rotate the tool electrode to speeds approximating 10,000 rpm or greater. Electrode diameters in the microns are possible, and can be used for producing micro holes or other shapes in thin electrically conductive materials. The most common size range for Micro EDM can be from 20 μm to 250 μm, and such machines can routinely drill 10 μm to 200 μm with an accuracy of ±1-2 μm. Typically, the very small nature of this work requires the aid of a microscope to accomplish.

Another type of EDM process is a Mole EDM, which is a highly specialized EDM process having the ability to machine a curved path or tunnel through a work piece. This process was first referred to as “Mole EDM” in that the electrode functions like a mole digging a tunnel into the ground. The Mole EDM electrode shape is typically a bar-like construct which can be bent and a shape memory alloy is used as an actuator. An ultrasonic wave can be used to detect the form of tunnels machined by this process.

There are many advantages in using EDM as a manufacturing technique for creating implant components, including: (1) the ability to manufacture complex shapes that would otherwise be difficult to produce with conventional cutting tools; (2) EDM techniques can cut extremely hard materials to very close tolerances; (3) EDM manufacture may cut very small implants where conventional cutting tools may damage the part from excess cutting tool pressure; (4) with EDM there is no direct contact between the tool and work piece, eliminating the need for excessive cleaning and/or removal of pyrogens; (5) EDM processing can create a good to mirror-like surface finish; and (6) very small diameter holes and other features can be easily drilled using various EDM techniques.

### Wire Edm & the Femoral Implant Blank

In the embodiments disclosed and discussed herein, wire EDM is one of the various EDM techniques that may be employed in combination with standard component machining to quickly and inexpensively create useful implant components from implant blanks

FIG. 5 depicts an isometric view of a fixture 160 for use with an implant blank that facilitates an initial step of “skimming” a reference and/or securement feature of the blank 10, which in this instance are the supports posts 140 and 142 of the femoral implant blank 10 of FIG. 1. This fixture 160 secures the implant in a desired position and alignment, which may not be an “optimal” or exact position relative to the EDM equipment, but rather the implant is positioned in a reasonably accurate position relative to the fixture 160 and immobilized for the subsequent EDM processing step. This arrangement allows the blank to be quickly placed within the EDM processing enclosure, with the blank positioned within a desired range of positioning and/or orientation error (i.e., within 0.5 mm of a desired location and within a few degrees of a desired orientation) within the fixture. This arrangement also facilitates the use of a single fixture or fixture type to be used to secure blanks of various shapes and sizes, depending upon which blank is selected for processing.

The fixture 160 permits a technician or operator to secure the implant therein and thus provides a stable platform to secure the femoral implant blank 10 within the EDM processing equipment. The implant blank may be secured by a locking mechanism 170 that may include a screw thread or other tightening feature to pin the implant tightly to the fixture during the EDM process, thereby restrict significant movement. The locking mechanism 170 may be designed as a vise, as a press fit, as a dove tail, or as any other preferred locking mechanisms known in the industry. The fixture 160 allows the support posts 140 and 142 of the implant 10 to extend beyond the surface of the fixture 160, whereby a wire EDM processing step can be employed to cut the posts 140 and 142 to a precise thickness and planar orientation on the anterior faces 182 and 184 (see FIG. 6B) and posterior faces 183 and 185 (see FIG. 6B) of the posts 140 and 142. In addition, the bottom surfaces 188 and 189 of the posts 140 and 142 can be cut to a known position and alignment. In the disclosed embodiment, this cutting operation will create a film or “skin” 180 (see FIG. 6A) from the implant blank support posts 140 and 142, aligning the exposed anterior, posterior and bottom surfaces of the support posts and creating a known “datum” or orientation point(s) relative to the remainder of the blank 10.

The EDM support post fixture 160 may accommodate varying sizes of the implant blanks. The fixture 160 may incorporated a variety of screw holes 190 where support plates or other features can be connected and/or expanded in a known manner to accommodate the various implant blank dimensions, sizes and/or widths. In one preferred embodiment, a given fixture may accommodate up to 3 different size blanks, which could be referred to as small, medium and large blanks (not shown). In various alternative embodiments, the support plates or other features could be slidably attached (not shown) to accommodate fractional sizes, if preferred.

FIG. 6A depicts an isometric view of a material “skin” 180 that has been removed from the support posts of a femoral blank via EDM processing using the EDM support post fixture 160 of FIG. 5. Once the skin 180 is removed from the implant blank, the anterior, posterior and bottom surfaces of the posts are precisely cut (see FIG. 6B) and one or more (or various combinations thereof) of these surfaces can be employed in subsequent steps as securement features and as datum for a variety of subsequent processing operations. For example, the precisely cut support post surfaces can be used as axis datums useful during the remaining wire EDM and machining operations. In various additional embodiments, the precise cuts on the support posts and the interrelationships there between may be useful during subsequent manufacturing operations to determine if the implant material is grossly deforming, relaxing or otherwise altering in some manner.

FIG. 7 depicts an isometric view of an EDM fixture useful in a second cutting operation to skim or cut a medial/lateral profile of the skimmed femoral blank 250 of FIG. 6B. The second fixture 200 may include a variety of features, including a slidable pallet 210, a locking lever 220, a 3 R chuck 230, and/or a variety of other features known in the art. In the exemplary embodiment, the fixture 200 also includes a channel block 240, which is sized and configured to accommodate the skimmed support posts 140 and 142 of the skimmed implant blank 250. In one preferred embodiment, the support posts 140 and 142 can be cut in a slight dovetail fashion in the initial EDM operation, such that placement of the posts in the corresponding channel of the block 240 and tightening of one or more compression screws 245 (or other features) drawing the skimmed implant blank 250 into the block 240, securing it therein.

The second skimming operation can be performed to cut a medial/lateral profile of the implant design into the blank 250, although various other profile orientations could be accomplished in this manner. The second fixture 200 secures the blank 250 in an orientation such that the EDM wire (not shown) can cut completely through the blank and/or portions of the fixture 200 (if desired), without releasing the blank 250 from the fixture 200. The skimming process can create various approximations of complex outer and inner implant profiles that more closely approximate the femoral implant shape than the original blank profile. In this specific embodiment, the skimmed femoral implant blank 250 from FIG. 6B can be placed in a 90 degree vertical orientation within the channel block 240, which gives a vertically-positioned EDM wire the best access to the medial/lateral profile. The channel block allows the skimmed implant blank 250 to be tightly secured, and also provides one or more known reference points relative to one or more skimmed support posts 140 and 142, providing the EDM equipment with a known reference to the skimmed blank. The manufacturer can program the wire EDM machine to skim or otherwise cut the resulting medial/lateral profile of the implant blank 250.

FIG. 8A depicts an isometric view of an exemplary medial/lateral femoral profile material “skin” cut and removed using the EDM fixture of FIG. 7, and FIG. 8B shows the profiled implant blank 280 after this secondary EDM operation has been completed. The EDM processing has created an inner profile surface 290, which will eventually correspond to an inner, bone-facing surface of the implant component, and an outer profile surface 295, which corresponds to an outer, articulating (or joint-facing) surface of the implant. FIG. 8B also shows a central rib 300, the design for which has been programmed into the wire EDM cutting profile. In the disclosed embodiment, one or more securement pegs (not shown) can be formed from the rib 300 to help secure the implant component to the patient's underlying boney anatomical features, or any other designs can be used that may help align the implant on the patient's femur.

FIG. 9 depicts a side view of an EDM fixture arrangement that facilitates the removal of additional excess material from the anterior and posterior portions of the profiled implant blank 280 in a subsequent EDM processing operation. The fixture facilitates securement of the profiled blank 280 at an angle relative to the EDM wire, thereby allowing cutting of the posterior face features of the profiled blank 280 without cutting commensurate features in the anterior face of the profiled blank 280. In various preferred embodiments, the fixture may allow the profiled blank 280 to be secured at processed at a plurality of angles, and even allow for “flipping” or inverting of the profiled blank 280 prior to additional EDM processing, when desired.

In the embodiment of FIG. 9, the EDM fixture 305 includes a block platform 330, a securing vise 320, a 3 R chuck 230, and a rotatable pallet 310. The fixture 305 allows controlled articulation and/or rotation of the casting to position it to various desired angles for EDM cutting processes. The jaws 321 and 322 of the vise 320 can include a dovetail securing feature, to provide for securement of the corresponding dovetail features of the posts 140 and 142 of the profile blank 280 and act as datum and/or alignment features relative to the support posts 140 and 142. The rotatable pallet can be rotated or otherwise indexed to a variety of angles, including complete inversion of the platform 330 and vise 320.

The implant may be securely fastened within the securing vise 320, which in turn may be slidably movable on the block platform 320. In other embodiments, the manufacturer may decide to use alternative securing mechanisms, such as threaded fasteners, grips, press fits, and any other variable securement mechanisms known in the industry.

In one exemplary embodiment, the profiled blank 280 can initially be positioned at an angle of approximately 45 degrees, such as shown in FIG. 10, with a posterior portion 335 of the implant extending laterally outward from the fixture (not shown). Once in this desired position, the wire EDM machine can cut or otherwise remove excess material from the posterior side of the implant (i.e., processing the shaded section of the blank 280 in FIG. 10), with the vertical cutting wire 340 travelling along a predetermined cutting path (not shown), which avoids cutting the anterior side 345 of the implant in an undesired fashion.

In various alternative embodiments, cutting of unwanted material from the anterior side during this same operation is contemplated and may be desired, depending upon the implant design.

FIG. 11 depicts a side view of the profile implant blank after being inverted or rotated to an opposing 45 degree angle, with an anterior portion 345 of the implant extending laterally outward from the fixture (not shown). This may be accomplished by rotating the fixture in a desired manner, or by removing the profiled implant blank 280 from the EDM fixture, rotating it manually, and replacing it in the same or different fixture. Once secured into a desired second orientation, the wire EDM machine can cut or otherwise remove excess material from the anterior side 345 of the implant (i.e., processing the shaded section of the blank 280 in FIG. 11), with the vertical cutting wire 340 travelling along a predetermined cutting path (not shown), which avoids cutting the posterior side 335 of the implant in an undesired fashion.

In various embodiments, a single EDM fixture may be used to position the implant at approximately 45 degrees in each operation to complete the profile cutting steps. Alternatively, the manufacturer may use a fixture that is rotatable in various axis to allow material removal without requiring removal and re-securing of the implant blank, which may require reconfirming of various datum axis for ach operation. A fixture that allows rotation to cut both the anterior and posterior cut planes without requiring removal of the blank 280 from the fixture can potentially prevent additional errors and defects on the surface of the implant blank during the wire EDM process. After this final wire EDM step has been performed, the manufacturer may decide to conduct additional inspection of the various cut surfaces before proceeding to the next machining process or any other finishing processes.

It should be understood that various other angles for the implant blank may be used for processing the anterior and posterior portion of the implant blank, depending upon the specific design and configuration of the intended implant features. Moreover, the various angles for processing of the anterior and posterior sides of the implant may be unequal angles, as desired. In various alterative embodiments, the blank may be processed along a first plane (which may include one more straight, curved and/or complex cutting paths of the wire through the blank) and then the piece may be rotated or otherwise reoriented and then processed along a second plane (which may include one more straight, curved and/or complex cutting paths of the wire through the blank). A variety of such successive reorientations of the blank and subsequent EDM cuts can be accomplished as desired, depending upon the selected blank and the intended implant component design, including the use of 2, 3, 4, 5, 6, 7, 8, 9 or 10 or more reorientations and associated EDM cuts, as desired by the designer and/or manufacturer.

FIG. 12 depicts an isometric view of one preferred embodiment of a femoral implant blank 360 that has completed the initial wire EDM shaping processes described herein. This implant blank 360 approximates many of the eventual shapes, contours and/or features of the patient-specific implant, which includes incorporating many of the complex contact surface planes and contours required of an implant design to match and/or conform to the patient's anatomy. Generally, however, the shape of the blank 360 may crudely or approximately resemble the final implant shape (i.e., the shape may approach a near or net-near shape of the final implant), with many of the dimensions of the blank 360 slightly larger in dimension than the corresponding patient-specific objectives, thereby allowing for varying amounts of material removal during subsequent processing operations to create the final patient-specific implant features. In addition, because the EDM cutting process can create surface variations or roughness (depending upon specific EDM processes and speed/power of the machine), it can desirous to have at least a thin layer of material remaining on the blank surface after EDM processing for subsequent mechanical machining, milling, grinding and/or polishing of various surface layers, such as articulating surfaces of the implant component.

The various wire EDM operations previously described will permit processing of the blank to an approximate shape of the implant component in only a few operations, removing a significant amount of bulk material from the blank to facilitate subsequent conventional machining, drilling and/or milling of the blank 360 to create the final finished patient-specific implant component in a quick and accurate manner. Various embodiments of the wire EDM processes described herein may leave a few millimeters thickness of material on the vast majority of the various surfaces of the blank (when compared to the final implant component dimensions), which can be removed relatively easily by conventional CNC tooling. By significantly reducing the amount of bulk material to be removed in subsequent machining operations, the manufacturing techniques described herein significantly increase the speed at which CNC or other machining equipment can subsequently process the cut blank to a finished implant. Moreover, by significantly reducing the amount of material required to be machined, the described techniques significantly reduce the amount of wear experienced by the various CNC tooling (or other equivalent processing equipment), potentially reducing and/or eliminating the need for offsets that adjust for variations in tool geometry due to tool wear as well as reduce the need for replacement tools.

FIG. 13 illustrates a side view of the preferred embodiment of the implant blank 360, showing the various cut planes and medial/lateral profile obtainable by the EDM multi-step processes described herein. This view highlights various inner surface cut planes 370 formed during the wire EDM process, which corresponds to bone-facing surfaces in the final finished implant component. The cut planes preferably approach the final patient-specific implant dimensions, including approximations of the various M-L dimensions, condylar heights, implant thicknesses 390 and approximate contour edges 380.

FIG. 14 depicts a bottom view of the implant blank 360, showing additional complex contours 410 of the blank that approximate various patient-specific dimensions of the final patient-specific implant. This view also shows the support posts 140 and 142, as well as a centrally-located support post platform 400 that typically remains formed into the implant blank 360 during the initial wire EDM processing steps. In the exemplary embodiment, the support post platform 400 and associated support posts 140 and 142 can be employed to secure the blank 360 into the various fixtures. After the EDM processes have been completed, the support posts 140 and 142 and the support post platform 400 can be removed partially or in their entireties, or these structures can be retained to support the implant for various machining operations for forming the inner surfaces and/or bone-facing posts of the implant.

FIG. 15 depicts a posterior/anterior view of the implant blank 360. In both the posterior and anterior sides 440 and 420 of the blank 360, the various wire EDM cut planes can be seen to have created surface features that approximate the final desired dimensions of the patient specific implant component.

FIG. 16 depicts a top view of the implant blank, highlighting an intercondylar notch portion 430 as well as portions of the support post platform 400, which can be removed by later machining of the blank.

FIG. 17 depicts an isometric view of one embodiment of a trunnion fixture 460 that may be used for CNC machining of the implant blank 360 in subsequent processing steps. In this embodiment, the trunnion fixture consists of a 4^{th} axis table that allows at least two different fixtures to be mounted onto the table. This view highlights the 3 R chuck 230, the support center housing 470, the trunnion plate 480, placement points for a first fixture 490 and placement points for a second fixture 500 (which in this embodiment are both located on the trunnion plate 480). The 3 R chuck 230 can be used to connect a variety of fixtures or tools for securing blanks at various stages of processing, including those that may require rotation at various angles. The support center housing 470 may be used as a secondary mechanism to secure the fixtures, tools or implants that may be positioned in the first 490 or second 500 placement points of the trunnion plate 480. Also, the support center housing 470 may also be used for placement of other fixtures, tool or implants to assist with other steps in the finishing process. The trunnion plate 480 can be used as a stable platform to secure all fixtures and tooling, and may be rotated a desired amount to achieve various complex angles and/or axis requirements for manufacture of a desired implant component design.

FIG. 18 illustrates a side view of the trunnion fixture of FIG. 17. The trunnion fixture 360 may be similar to trunnion fixtures that are commercially available from various suppliers, including Tsudakoma Rotary Table manufacturer. The trunnion fixture 460 in this embodiment may employ a rotary union 510 to rotate or otherwise manipulate the implant blank during machining of the various implant surfaces.

FIG. 19 depicts a fixture 520 for securing the implant blank 360 for machining of the inner implant surface. The fixture 520 can include a block platform 330 and a securing vise 320. In various embodiments, the fixture can be securely fastened to a trunnion fixture 460 (see FIG. 17) to allow 4 axis rotation of the blank 360 and machining of the various accessible surfaces of the implant component to the specifications required for the patient. Other securing mechanisms that may be used may include threaded fasteners, grips, press fitting, and any other variable mechanism's known in the industry. Once secured, the manufacturer may program the CNC machine to machine or otherwise cut the remaining material on the accessible implant surfaces to match desired features and measurements of the final implant component. FIGS. 20 through 23 show various views of an implant blank 360 secured to the fixture 520.

FIG. 24 depicts a perspective view of one embodiment of a fixture 540 for holding a partially finished implant component for subsequent machining of various edge and outer or bone facing surfaces of the component. The fixture 540 can be attached to a 3 R chuck 230, such as depicted in FIG. 25, and the entire assembly may be securely fastened on a trunnion fixture, such as shown in FIG. 17. FIG. 25 also highlights a first fastening hole 550, a second fastening hole 570, and a series of custom cut planes 560 and 565 that may be designed and manufactured to match various finished inner surfaces of the patient specific implant that were previously created using combinations of the EDM wire cutting process and subsequent machining processes. The first fastening hole 550 may be designed in the fixture to closely match a first peg of the femoral implant (not shown), and the second fastening hole 570 can be designed slightly larger or elongated to accommodate a second peg of the implant, allowing for minute positioning changes when the implant (not shown) is secured onto the fixture 540. Once the implant pegs are aligned and positioned within both the first 550 and second 570 fastening holes, the technician may place a bolt or other securing mechanism from the opposite (or bottom side—not shown) to draw the implant towards the block and tightly fasten the implant to the fixture. FIG. 25 also highlights various custom cut planes 560 and 565 that have been formed onto the fixture to ensure that the implant properly fits said planes and does not move relative to the fixture 540 during the final machining, grinding and polishing processes. The custom cut planes can be derived using patient-specific anatomical data of the patient, and can be formed into the physical block (using a variety of manufacturing processes, which may include wire EDM processing), with the cut planes preferably corresponding to the intended cut planes of the surgical procedure for preparing the patient's anatomy for implantation of the implant component. In various embodiments, the various cut plane dimensions may be derived from a database library for approximate matching of the surfaces of the implant to the articulating surface fixture 540.

FIG. 26 depicts a side view of the fixture of FIG. 24, highlighting the differing heights of the fixture to accommodate medial 590 and lateral 580 inner surfaces of the implant. The heights and widths of these surfaces (as well as the various other custom cut planes 560 and 565) may be derived from implant dimensional data and/or surgical procedural planning data, as well as derived using the various images taken from the patient or from a database library for accurate machining and reduction or elimination of the blank sliding on the fixture. If desired, the fixture 540 can be used as a “check” or inspection piece to ensure the inner surfaces of the implant have been properly machined and prepared to fit the planned surgical modification of the patient's anatomy. FIGS. 27 through 29 depict multiple plan views of a machined implant blank 600 placed on the fixture 540.

FIG. 30 depicts an isometric view of the fixture 520 with an attached implant blank 360 and a fixture 540 with an implant 600 mounted on a trunnion fixture 460. In various embodiments, a manufacturer may opt to conduct various machining steps simultaneously. FIGS. 31 and 32 depicts various additional views of the mounting arrangements of FIG. 30.

FIG. 33 depicts a top view of an exemplary bone model fixture 610 that may be used for inspection of the final resulting implant. The bone model fixture 610 can be a disposable fixture that is tailored to a patient specific morphology. The data to design such a fixture may be derived from the patient-specific images that were used for surgical planning purposes or may be derived from a database library. Using such a patient-specific fixture can confirm that the various manufacturing processes described herein have met desired specifications.

The bone model fixture 610 may include two mounting holes 620 to align the resulting implant into the designed positioned and/or orientation, with the mounting holes reflecting intended bore holes (not shown) to be drilled in the patient's femur to accommodate the anchor posts of the implant. Alternatively, the manufacturer may include other features to assist with alignment or placement on the bone model fixture 610, such as a channel, or guiding edges (not shown). Also, the bone model fixture 610 may include patient-specific cut planes 630 and a bone model intercondylar notch 640 to match the resulting implant cut planes and notch for a seamless fit.

FIG. 34 depicts a side view of the bone model fixture of FIG. 35, with an attached final machined implant 600.

The bone model fixture 610 may be made from a variety of materials that may help with sterilization, cleanliness, and reduction of pyrogens, should the inspection be performed in cleanroom setting. In one preferred embodiment, the bone model fixture 610 may be made using SLA rapid prototype modeling techniques. Such material may be porous and can be easily machined and disposed of after the inspection for the patient-specific implant has been performed. Also, should the manufacturer decide to make a bone model fixture 610 that is not patient specific and/or disposable, the manufacturer may use a variety of metals, such as aluminum, steel, cobalt, metal alloys or combination thereof to have the fixture sterilizable and reusable. However, other materials may be contemplated even if the fixture is disposable or nondisposable, such as plastics, delrin, or various combinations of plastics and metals can be used.

FIGS. 35A through 35D depict a final patient-specific implant component 800 for an individual patient's anatomy that has been manufactured using the various EDM and machining techniques described herein. The anterior section 810 of the implant 800 includes cement pockets 805 and/or other known features machined into an inner side 820 of the implant. The inner side 820 also includes anchoring posts 830 and 840 for securing the implant 800 to the patient's anatomy. If desired, one of more of the surfaces of the cement pockets 805 may be roughened, texturized, bead blasted, and or grit blasted (or finished in other manners) to provide a surface that adheres well to surgical cement and/or which allows bony-ingrowth into the implant. Similar features have been machined into inner surfaces of the center section 813 and posterior section 817 of the implant 800.

The outer side 825 of the implant 800 can include smoother finished surfaces that can function as articulating surfaces for interaction with the patient's natural anatomy and/or with corresponding surfaces of another implant component (i.e., a tibial or patellar implant component). These surfaces can be smooth, continuous surfaces that may be polished to a high-gloss or mirror-like finish and can be shaped to provide a smooth, gliding action for articulation of the implant component in a known manner. In one exemplary embodiment, the various EDM and machining processes described herein can create articulating surfaces of the implant having thicknesses slightly larger than a final desired dimension after polishing, such as thicknesses of between 0.017 inches to 0.019 inches of extra material that can be removed during a final polishing step.

### Materials

Any material known in the art can be used for any of the implant systems, tools and fixtures, and components described in the foregoing embodiments, for example including, but not limited to metal, metal alloys, combinations of metals, plastic, polyethylene, cross-linked polyethylene's or polymers or plastics, pyrolytic carbon, nanotubes and carbons, biologic materials, or any combination thereof. In addition, any of the rapid prototype materials may be used for any of the tools or fixtures required during the EDM or machining processes.

Any fixation techniques and combinations thereof known in the art can be used for any of the implant systems and component described in the foregoing embodiments, for example including, but not limited to cementing techniques, porous coating of at least portions of an implant component, press fit techniques of at least a portion of an implant, ingrowth techniques, etc.

### Additional Embodiments

The embodiments discussed in this specification are exemplary, and many additional embodiments, features and combinations of features not discussed in this specification are possible. The foregoing embodiments are therefore to be considered illustrative, and are not intended to limit the scope of the specification, including, any equivalents.

What is claimed is:

1. A method of making a patient-specific implant component, the method comprising:
receiving patient-specific information;
deriving a design for the patient-specific implant component based, at least in part, on the patient-specific information;
selecting a suitable and/or available implant blank, the selecting based, at least in part, on the derived design;
cutting one or more support posts resulting in one or more cut support posts that include one or more surfaces that are aligned in a known orientation relative to the remainder of the blank, the cutting of the one or more support posts comprising a wire EDM process;
securing the blank relative to a cutting apparatus using the one or more cut support posts;
cutting the secured blank to produce a medial and/or lateral profile that is based, at least in part, on the derived design;
removing the one or more cut support posts from the blank; and
machining the blank using a conventional machining process to cut remaining material of the blank to produce surfaces that match desired features and/or measurements of the derived design.

2. The method of claim 1, wherein the selected implant blank includes the one or more support posts.

3. The method of claim 1, wherein the one or more cut support posts provide one or more predetermined axis relative to one or more surfaces of the blank and/or the derived implant design.

4. The method of claim 1, wherein the step of cutting one or more support posts includes cutting each of the one or more support posts to a predetermined thickness and planar orientation on an anterior face and posterior face of the one or more posts, thereby aligning the anterior and posterior faces of the one or more cut support posts and thereby creating one or more known orientation points relative to the remainder of the blank.

5. The method of claim 1, wherein the step of cutting one or more support posts includes initially cutting each of the one or more support posts using a wire EDM process to roughly correspond to predetermined dimensions of the one or more cut support posts.

6. The method of claim 5, wherein the step of cutting one or more support posts further includes subsequently cutting each of the one or more support posts using a wire EDM process to more precisely correspond to the predetermined dimensions of the one or more cut support posts relative to the rough correspondence of the initial cutting.

7. The method of claim 1, wherein the step of cutting the secured blank to produce a medial and/or lateral profile comprises cutting posterior portions of the blank to produce features of the posterior side of the implant design with the blank secured in a first position.

8. The method of claim 7, wherein the step of cutting the secured blank to produce a medial and/or lateral profile comprises cutting anterior portions of the blank to produce features of the anterior side of the implant design with the blank secured in a second position, the second position distinct from the first position.

9. The method of claim 1, wherein the step of cutting the secured blank to produce a medial and/or lateral profile comprises forming one or more inner, bone-facing surfaces and/or one or more outer, joint-facing surfaces based, at least in part, on the implant design.

10. The method of claim 1, further comprising performing a skim cut of the secured blank by applying a wire EDM technique at a lower power setting and/or with a lower pressure flush relative to wire EDM techniques applied in previous steps.

11. The method of claim 1, wherein one or more of the cutting or machining steps is configured to produce a blank having one or more articulating surfaces that have a dimension slightly larger than a corresponding desired dimension of the derived implant design.

12. The method of claim 11, further comprising polishing the blank subsequent to the one or more cutting or machining steps such that extra material is removed and the one or more articulating surfaces have a dimension matching the corresponding desired dimension of the derived implant design.

13. The method of claim 1, wherein one or more of the cutting steps is configured to create patient-specific cut planes in the blank that correspond to cut planes of the surgical procedure derived, at least in part, from the patient-specific information.

14. A method of making a patient-specific implant component for use in a surgical procedure, the method comprising:
receiving patient-specific information;
deriving a design for the patient-specific implant component based, at least in part, on the patient-specific information;
initiating manufacturing of a primary patient-specific implant component, wherein the manufacturing of the primary patient-specific implant component comprises employing one or more standard manufacturing techniques according to specifications to produce an implant component matching the derived design for the patient-specific implant component;
designating an implant blank as a backup, based, at least in part, on the derived design;
if the primary patient-specific implant component is successfully produced such that it satisfies appropriate inspection parameters, providing the primary patient-specific implant component for use in the surgical procedure; and
if the primary patient-specific implant is not successfully produced and/or does not satisfy appropriate inspection parameters, providing a backup patient-specific implant component for use in the surgical procedure,
wherein, the patient-specific implant component includes at least one joint-facing surface having a curvature derived, at least in part, from the patient-specific information,
wherein the backup patient-specific implant component is manufactured from the designated implant blank according to steps comprising:
providing the implant blank designated as the backup;
cutting one or more support posts utilizing a wire EDM process to produce one or more cut support posts;
securing the blank relative to a cutting apparatus using the one or more cut support posts;
cutting the secured blank to produce a medial and/or lateral profile that is based, at least in part, on the derived design;
removing the one or more cut support posts from the blank; and
machining the blank using a conventional machining process to cut remaining material of the blank to produce surfaces that match desired features and/or measurements of the derived implant design.

15. The method of claim 14, wherein the one or more standard implant manufacturing techniques comprise a manufacturing technique selected from the group consisting of casting, forging, CNC machining, drilling, cutting, milling, lathing, abrading, and combinations thereof.

16. The method of claim 14, wherein the primary patient-specific implant not successfully being produced comprises a fracture or failure in material comprising the primary patient-specific implant.

17. The method of claim 14, wherein the primary patient-specific implant not successfully being produced comprises a sufficient discrepancy between at least a portion of the primary implant produced and a corresponding portion of the derived design.

18. The method of claim 14, further comprising selecting the implant blank from a database of blanks, the selecting based, at least in part, on the derived design.

19. The method of claim 14, wherein manufacturing the backup implant component from the designated implant blank is subsequent to a determination that the primary implant will not be successfully produced.

20. The method of claim 14, wherein the designating an implant blank as a backup is prior to a determination that the primary implant will not be successfully produced.
